(19) United States
(12) Patent Application Publication
(10) Pub. No.: US 2025/0248701 A1
(43) Pub. Date: Aug. 7, 2025

(54) SKIN FLAP RETRACTION DEVICE

(71) Applicant: DESSINRX HEALTHCARE PVT. LTD, Hyderabad (IN)

(72) Inventors: Gorrepati ROHITH, Hyderabad (IN); Gorrepati VIVEK, Hyderabad (IN); Devyani SINGHI, Hyderabad (IN)

(21) Appl. No.: 18/855,647

(22) PCT Filed: Apr. 10, 2023
(86) PCT No.: PCT/IN2023/050344

§ 371 Date: Oct. 10, 2024

(51) Int. Cl. A61B 17/02

(52) U.S. Cl. CPC A61B 17/0206; A61B 17/0218

(57) ABSTRACT

Skin flap retraction device. The self-retaining retractor device (100) can provide appropriate, adequate, and dynamic traction during a surgical procedure that involves skin flap elevation. The self-retaining dynamic surgical retractor device (100) employs a precurved detachable solid block (20) and malleable arm assembly (22 and 24) that can be used for various surgeries involving raising skin flaps and places that need a dynamic yet gentle traction, for example, during a mastectomy procedure, which typically requires raising of skin flaps.

### TECHNICAL FIELD

The present invention relates medical and surgical devices. The present invention also relates to methods and systems for skin flap retraction. Further, the invention relates to surgical retractors. Further, the present invention specifically relates to an improved skin flap retraction device for providing adequate and dynamic traction in skin flap elevation and surgical procedures.

### BACKGROUND OF THE INVENTION

A skin flap is healthy skin and tissue that is partly detached from an underlying structure and a chunk of diseased tissue is removed or is otherwise usually moved to cover a nearby wound. A skin flap may contain skin and fat, or skin, fat, and muscle. The subcutaneous layer (or deeper layer depending on the type of surgery) is incised and the skin along with subcutaneous fat is lifted off from the underlying tissue in a surgical procedure. The edge(s) of the skin is retracted, and a vertical/inclined traction is provided by the assistants using a skin-hook retractor device. The entire underlying tissue will be separated from the skin flap using an electrocautery/harmonic scalpel device depending on the surgeon's preference.

It is to be noted that maintaining the traction in the skin is important in the surgical procedures without which, the plane of dissection will be missed, resulting in thinner or thicker flaps. Either of these flaps is prone for ischemia due to inadequate blood supply resulting in flap necrosis. The discussed problem can be very severe in surgeries such as ilio-inguinal block dissection, where the rates soar as high as 27-85%. Also, there are chances of leaving large amounts of fat globules attached to the skin flap resulting in postoperative seroma formation. The complication is seen in 30% of the patients who undergo mastectomy in which skin flaps are elevated.

Surgical retractors are generally designed for providing adequate visual and operative access to the surgical field for the operating surgeon. Traditional retraction systems usually require one or more assistants to retract the tissues away from the surgical field for adequate exposure.

Rigid hand-held traditional retractors are the most used retractors in almost all the surgeries in one step or the other. Such devices require continuous manipulation in terms of direction and force applied. In most of the cases, the assistant, although a skilled surgeon, would not get an adequate exposure to the surgical field. It is a tedious task and many a time, interferes with the operating surgeon's efficiency thereby prolonging operative time. Tension applied at various places would be unequal resulting in a faulty thickness of the flap, resulting in flap necrosis and other complications.

Various self-retaining retractors have been designed and developed in the recent past but have not been widely used due to the limitations the design and various other reasons. Majority of the prior art devices are mounted over the operating table to enable retraction. But the size and rigid framework of such prior art devices make them cumbersome to use in surgical procedures. Such devices are further complicated and require multiple parts. Problems such as need for frequent realigning and repositioning when dislodged precluded their success. Additionally, human breast varies in its size, shape, volume, and laxity. It is difficult for a single rigid retracting assembly to be used in all the cases with equal ease and efficiency.

In one embodiment of prior art a manually held skin-hook retractor is proposed to give traction force on the skin flap. The operating surgeon gives counter-traction with his left hand while dissection is carried out with an energy device held in his right hand. The proposed device requires the assistant to hold the skin hooks for a prolonged period of time.

In another embodiment of prior art, eyebrow retractors have also been utilized in breast surgery, which have a stainless-steel retractor ring and elastic hooks. However, the retractor ring is rigid and cannot be used only for the upper flap. It is difficult to use as it has a fixed structure, but the structure of the breast varies from person to person to a large extent. So, thereby manual retraction using assistants is the preferred method for skin flap rising.

Based on the foregoing, it is believed that a need exists for an improved self-retaining retractor system that can provide appropriate, adequate and dynamic traction during a surgery that involves skin flap elevation. Also, a need exists for an improved skin flap retraction device for providing adequate and dynamic traction in skin flap elevation and surgical procedures, as described in greater detail herein.

### SUMMARY OF THE INVENTION

The following summary is provided to facilitate an understanding of some of the innovative features unique to the disclosed embodiment and is not intended to be a full description.

It is, therefore, one aspect of the disclosed embodiments to provide for an improved skin retractor device and method.

It is another aspect of the disclosed embodiments to provide for an improved self-retaining retractor system that can provide appropriate, adequate and dynamic traction during a surgery that involves skin flap elevation.

It is further aspect of the disclosed embodiments to provide for an improved skin flap retraction device for providing adequate and dynamic traction in skin flap elevation and surgical procedures.

A skin flap retraction device (100) is disclosed herein. The self-retaining retractor device (100) can provide appropriate, adequate, and dynamic traction during a surgical procedure that involves skin flap elevation. The self-retaining dynamic surgical retractor device (100) employs a precurved solid block (20) and malleable arm assembly (22 and 24) that can be used for various surgeries involving raising skin flaps and places that need a dynamic yet gentle traction, for example, during a mastectomy procedure, which typically requires raising of skin flaps.

The skin flap retractor device (100) comprises a central pre-curved solid block (20) with a first malleable (or deformable or bendable) arm portion (22) and a second malleable arm portion (24). The first malleable arm portion (22) and the central pre-curved solid block (20) are coupled at the first respective end (18) of the first malleable arm portion (22) and the first respective end (16) of the central pre-curved solid block (20) thereof via. a first couple assembly (28) and is secured using a screw (32) or any kind of fasteners.

The second malleable (or deformable or bendable) arm portion (24) and the central pre-curved solid block (20) are coupled to one another at the first respective end (26) of second malleable arm portion (24) and the second respective end (14) of the central pre-curved solid block (20) thereof via. a second couple assembly (30) and secured using a screw (34). The device (100) is mounted on the base plate (36).

### BRIEF DESCRIPTION OF THE DRA WINGS

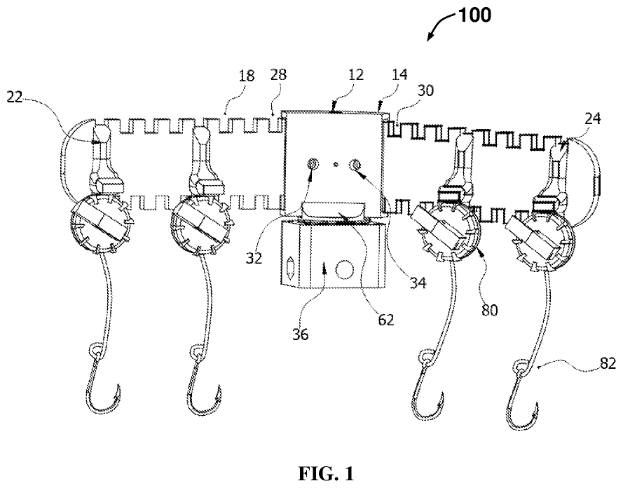

FIG. 1-9 illustrate embodiments of the skin flap retraction device (100) of the present invention and its various components.

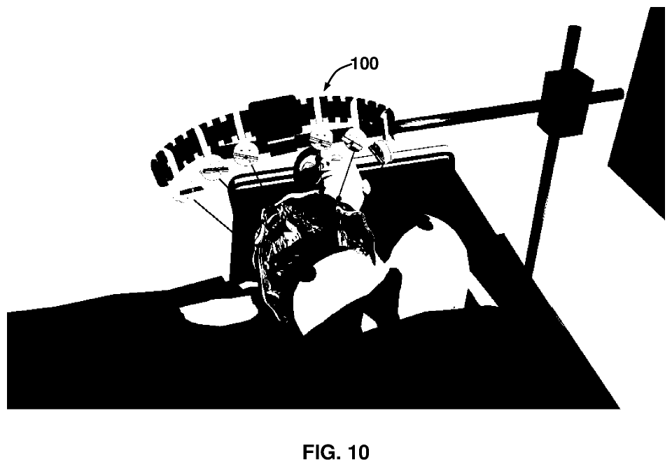

FIG. 10 illustrates use of the skin flap retraction device (100) of the present invention for skin flap retraction during surgery.

### DETAILED DESCRIPTION

The values and configurations discussed in these non-limiting examples can be varied and are cited merely to illustrate at least one embodiment and are not intended to limit the scope thereof.

The embodiments now will be described more fully hereinafter with reference to the accompanying drawings, in which illustrative embodiments of the invention are shown. The embodiments disclosed herein can be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. As used herein, the term “and/or” includes all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Proposed Technical Advancement: The proposed skin flap retractor device (100) seeks to achieve an improved device (100) which mitigates or reduces the problems associated with elevation of skin flaps in a surgery. The device (100) further obviates the need for manual retraction by an assistant surgeon thereby improve the surgeon's exposure to the ongoing surgery. The device (100) provides a simple yet effective retractor with improvised dynamic retractor with self-retaining properties.

Detailed Description of the Invention: Referring to FIGS. 1-3, a retractor (100) is depicted. This retractor assembly (100) includes a precurved detachable central part (20) with a first malleable (deformable or bendable) arm portion (22) and a second malleable arm portion (24). The first malleable arm portion (22) and the central pre-curved solid block (20) are coupled to one another at the first respective end (18) of the first malleable arm portion (22) and first respective end (16) of the precurved detachable central part (20) thereof via. a first couple assembly (28) and is secured using a screw (32).

The second malleable (deformable or bendable) arm portion (24) and the precurved detachable central part (20) are coupled to one another at the first respective end (26) of the second malleable arm (24) and second respective end (14) of precurved detachable central part (20) thereof via. a second couple assembly (30) and secured using screw (34). The entire apparatus is mounted on the base plate 36. The arcuate shape of the retractor assembly 10 can be best appreciated in FIG. 1.

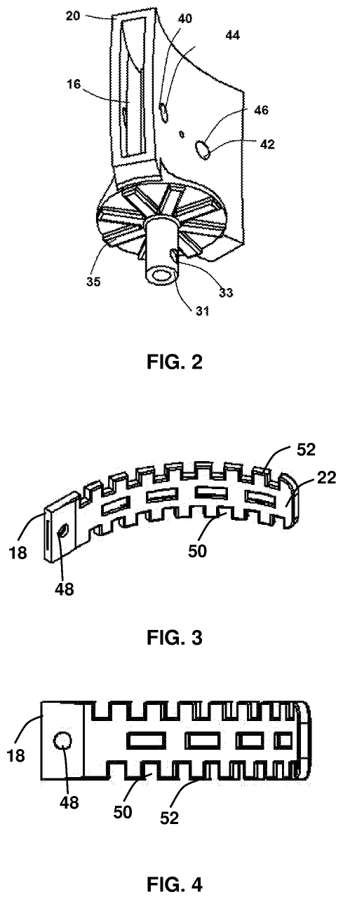

The precurved detachable central part (20) can be best appreciated in FIG. 2. The precurved detachable central part (20) has a rotatable shaft coupling system (38) at its base and a base plate with one or multiple force stoppers (61) on its superior surface which can accommodate the rotatable shaft coupling present on the base of the central part. On either of its sides, it has slots (14) and (16) that can accommodate malleable arms (22 and 24). On its posterior aspect, two holes (40 and 42) are provided that are used to secure the malleable arms (22 and 24) to the central shaft (20) using screws (32 and 34). Circular threading (44 and 46) with equal pitch throughout its length is engraved on their inner aspect. The pre-curved central part has peg like elevation (31) with a key hole (33) and one or more rotational stoppers (35) The central pre-curved solid block (20) is preferably made of stainless steel or surgical grade plastic, a titanium alloy, or the like. Other suitable material with the required rigidity and strength can also be used.

The first and second malleable arms (22 and 24) are preferably relatively flat and assume an arc shape (FIGS. 3 and 4) but they may, however be fabricated to other shapes resulting in semi-circular configuration, irregular configuration, or other configurations.

Each arm (22 and 24) has one end flattened (18 and 26) that can be accommodated into the sockets (16 and 14) on the central pre-curved solid block (20) respectively. In the centre of flattened ends (18 and 16), there is a hole (48) that can be used to immobilize the arm and central shaft assembly using screws (32 and 34). Each malleable arm portion (22 and 24) has is preferably made of stainless steel, copper, or the like. Other material with sufficient strength and thickness which provides both stability and malleable property to the instrument can also be used.

The retractor assembly (100) includes plurality of pitches or notches (50) made on the first and second malleable arm portions (22 and 24) (FIGS. 3 and 4). These notches or pitches (50) are equally spaced about the malleable arms (22 and 24) on both their upper and lower edges (52 and 54). The notches or pitches are used to affix or clip the torsion spring systems (e.g., torsion spring systems (70) that are discussed below), onto the arm portions (22 and 24).

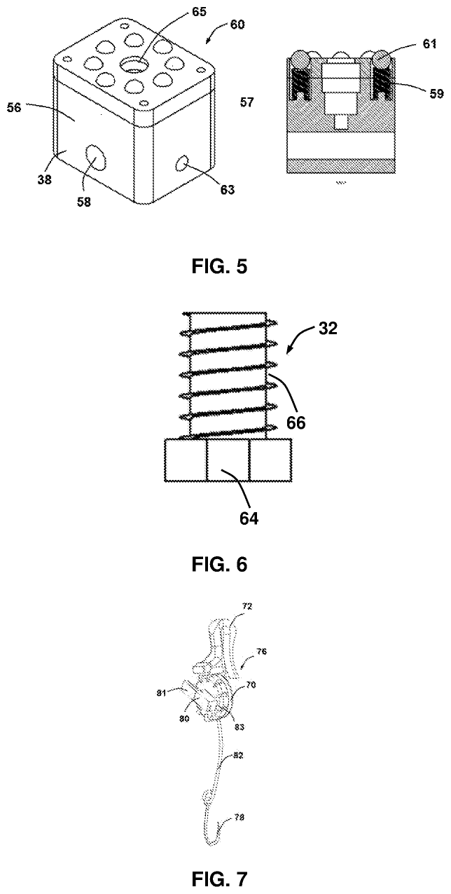

The retractor assembly (100) has a base plate 36 (See FIG. 5) on which it rests. It has a body (56) with one or more spring (59) and ball (61) sets and a cap (57). There is socket (65) which is passing through cap into body and which is complementary to the peg (31) on the base of the pre-curved central part (20). The central part (20) can be placed facing any direction by utilizing the peg and socket joint (62) formed by the base plate and the pre-curved central part (20). The body (56) necessarily has a central circular hole (58) extending throughout its length. This has a central hole which snuggly fits and glides over the support metallic arm that is used to mount the retractor and rigidly secured to the operating table. A knob screw can be placed in the threaded holed (63) which is perpendicular to the central hole (58) of the body (56) to tighten the assembly on the metallic arm.

The malleable arms (22 and 24) are attached to the pre-curved central part (20) using screws (32 and 340 (FIG. 6). Each of it has a head (64) and a shank (66) with threading throughout its length with equal pitch.

FIG. 7 depicts a spiral torsion spring system (70) that is used to elevate the skin flap. It has an elastic/rigid cable (82) with one end of it attached to a torsion spring system (76) and a metallic hook (78) on the other end. The hook (78) can be inserted into the subcuticular layer of the skin preventing its dislodgement. The spring system has a tension key (80) on its flat surface which can be used to increase or decrease the tension the coil at a given particular point of time. One or more than one unidirectional stopper (83) is located on the anterior surface of the spring torsion system to hold the tension at a given point. The split key (81) can be lifted up and rotated anti clockwise to decrease the tension in spring system,

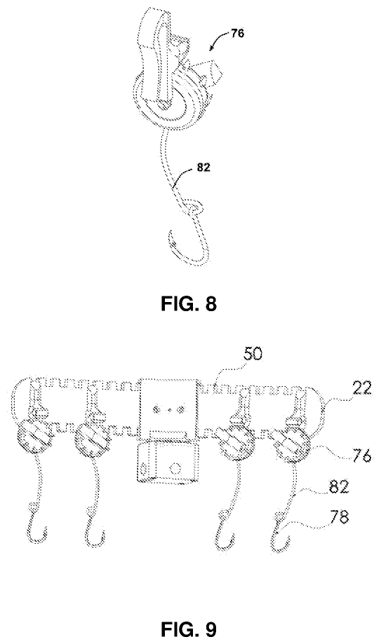

FIG. 8 represents the spiral torsion spring apparatus (80) with which variable stress and length can be provided to the skin flap. The spiral torsion spring system can be reversibly attached to the malleable arms at pitches or notches. The screw assembly attachment to the malleable arm is depicted in FIG. 9.

While FIG. 7 illustrates a spring torsion spring system 70, it would be understood that any other resilient component, including linear springs, or elastic threads or any other spring-based system could be used to implement similar functionality.

Working of the Invention: Referring to the FIG. 10, the retractor assembly (100) can be mounted on the operating table from the assistant's end, using an elongated support arm which is preferably made of stainless steel, carbon fibre, titanium alloy, or the like.

A surgical incision was depicted over the patient's breast. The upper flap edge (74) was being retracted using elastic slings spiral torsion spring coil mechanism (70). The slings are suspended from the notches using a screw clip apparatus (72). Referring to FIG. 3, the retractor assembly 10 can be rotated about the base plate 38 and the surgery on the lower flap can be carried out with ease.

It would be understood that during surgery, the retractor assembly can provide continuous dynamic retraction for rising skin flaps during surgery. The device can be suspended from the assistant's end using a solid rod that can be attached to the operating table. The device enables the surgeon to perform the surgery irrespective of the size, shape, volume or laxity of the organ of interest in a safe manner without the necessity of an assistant surgeon. To achieve this, the two malleable arms can be bent in either inward or outward direction at any part of their length. This enables the surgeon to manipulate the size and angle of the retractor and thereby facilitating its use in almost all kinds of surgeries. The central solid block provides the strength and stability to the retractor system. The device can be comfortably used in raising both the upper and lower flaps during mastectomy. To enable this, there is a peg and socket joint provided at the base of the midpoint of the central precurved solid block which can be rotated about the shaft of the on the baseplate which is also used to mount the retractor on the operating table. The device can provide dynamic and continuous traction by the elastic slings/cable that are is passed from the spiral torsion system attached to the notches or pitches present on the edges of the malleable arms. The tension delivered to the skin flap can be dynamically changed by manipulating the torsion delivered by the spiral torsion system. The tension generated can be maintained as well as changed dynamically using the tension key and unidirectional stoppers that are provided over the spiral torsion springs systems. As the rising of the flap progresses towards the retractor assembly, the spiral torsion spring recoils elastic sling contracts and provides continuous traction in that direction. Thus, this obviates the need for a manual retraction by an assistant surgeon.

Based on the above, it will be understood that the retractor assembly can be mounted on the assistant's end which is self-retaining and which doesn't require assistant surgeons. The device has malleable arms that can be contoured to the size or shape of the organ of interest. The device can be rotated about the base plate and can be used to operate on both upper and lower flaps. The device can be suspended directly above the operating field and so, vertical traction can be provided instead of horizontal traction. The device can provide uniform dynamic traction using the elastic slings/cables attached to the spiral torsion system throughout the length of the skin flap that can help in rising flaps of adequate and uniform thickness.

Based on the above, it will be understood that retractor assembly of the present invention provides multiple advantages over prior art solutions, including: offering a self-retaining retraction system (which needs no assistant surgeon). enables uniform traction force can be applied throughout the length of the skin flap at a given point of time; enables vertical traction can be applied by suspending the retractor directly above the surgical field; allowing the surgeon to operate freely from the other side (lower side when the upper flap is being raised) due to the arcuate shaped design; the retraction system can be suspended on the assistant surgeon's end; the malleable arm design with which the length can be modified based on the size and contour of organ of interest; the dynamic spiral torsion system with which the length and tension delivered to the skin flap via the elastic sling/cables can be dynamically manipulated.

While the exemplary embodiments of the present invention are described and illustrated herein, it will be appreciated that they are merely illustrative. It will be understood by those skilled in the art that various modifications in form and detail may be made therein without departing from or offending the spirit and scope of the invention as defined by the appended claims. Additionally, the invention illustratively disclose herein suitably may be practiced in the absence of any element which is not specifically disclosed herein—and in a particular embodiment that is specifically contemplated, the invention is intended to be practiced in the absence of any one or more element which are not specifically disclosed herein.

I/We claim:

1. A skin flap retraction device, comprising of:
a self-retaining retractor device (100) which can provide appropriate, adequate, and dynamic traction during a surgical procedure that involves skin flap elevation wherein the self-retaining dynamic surgical retractor device (100) employs a precurved detachable central precurved solid block (20) and malleable arm assembly (22 and 24) that can be used for various surgeries involving raising skin flaps and places that need a dynamic yet gentle traction wherein the central precurved solid block (20) is curved in shape with a radius of curvature based on the organ of interest wherein the curvature provides more flexibility for the self-retaining retractor system (100).

2. The device as claimed in claim 1 wherein the self-retaining retractor device (100) comprising of a precurved detachable central part (20) with a first malleable (or deformable or bendable) arm portion (22) and a second malleable arm portion (24) wherein the malleable arms (22 and 24) of the retraction system (100) can be bent in any shape based on the organ shape or in the shape of the incision and the retraction force can be delivered to the area of interest from any desired angle or direction.

3. The device as claimed in claim 1 wherein the precurved detachable central part (20) comprises a pin on the top, the central part can be easily interchangeable according to the needs of the surgeon, surgery and the body habitus of the patient.

4. The device as claimed in claims 1 and 2 wherein the first malleable arm portion (22) and the precurved detachable central part (20) are coupled at the first respective end (18) of the first malleable arm portion (22) and the first respective end (16) of the central precurved solid block (20) thereof via. a first couple assembly (28) and is secured using a screw (32) wherein the second malleable (or deformable or bendable) arm portion (24) and the central solid block (20) are coupled to one another at the first respective end (26) of second malleable arm portion (24) and the second respective end (14) of the central solid block (20) thereof via. a second couple assembly (30) and secured using a screw (34). The device (100) is mounted on the base plate (36).

5. The device as claimed in claims 1 and 2 wherein the precurved detachable central part (20) with slots (14) and (16) that can accommodate malleable arms (22 and 24) secured by malleable arms (22 and 24) comprises a rotatable shaft coupling system (38) at its base and a base plate with one or multiple force stoppers (33) on its superior surface which can accommodate the rotatable shaft coupling present on the base of the central part.

6. The device as claimed in claims 1 and 2 wherein the retractor assembly (100) comprises a plurality of pitches or notches (50) made on the first and second malleable arm portions (22 and 24) wherein the notches or pitches (50) are equally spaced about the malleable arms (22 and 24) on both their upper and lower edges (52 and 54) wherein the notches or pitches are used to affix or clip the torsion spring systems onto the arm portions (22 and 24).

7. The device as claimed in claims 1 and 2 wherein the retractor assembly (100) has a base plate (36) on which it rests wherein a body (56) and a socket (65) on its upper surface which is complementary to the peg (33) on the base of the central solid block (20).

8. The device as claimed in claim 1 wherein a spiral torsion spring system (70) is used to elevate the skin flap wherein the spiral torsion spring system (70) has an elastic sling/cable (82) with one end of it attached to a torsion spring system (76) and a metallic hook (78) on the other end wherein the hook (78) can be inserted into the subcuticular layer of the skin preventing its dislodgement.

9. The device as claimed in claim 1 wherein the retractor system (100) comprises a coil mechanism (70) providing variable traction force with the help of a key (80) wherein the applied traction force can be maintained with the help of unidirectional stoppers (83) wherein the traction force can be decreased by lifting the split key (81) and releasing it for auto rotation in an anti-clockwise direction.

10. The device as claimed in claim 1 wherein the retractor device (100) can be mounted on a baseplate and can be rotated at 360 degrees wherein the position of the instrument can be altered intraoperatively without disconnecting from the baseplate wherein the position of the retractor with respect to the base plate will be held using the force stoppers placed on the base plate wherein the central shaft (20) can be placed facing any direction by utilizing the peg and socket joint (62) formed by the base plate and the central shaft (20) wherein the body (56) necessarily has a central circular hole (58) extending throughout its length.
